(19) United States
(12) Patent Application Publication
(10) Pub. No.: US 2024/0319167 A1
(43) Pub. Date: Sep. 26, 2024

(54) CELL INFORMATION ACQUISITION METHOD, AND PROGRAM

(71) Applicant: CANON KABUSHIKI KAISHA, Tokyo (JP)

(72) Inventors: TAKUSHI ICHINOO, Tokyo (JP); ICHIRO HARADA, Kanagawa (JP)

(21) Appl. No.: 18/612,818

(22) Filed: Mar. 21, 2024

(30) Foreign Application Priority Data
Mar. 22, 2023 (JP) 2023-045118

(51) Int. Cl. G01N 33/50; G01N 21/64

(52) U.S. Cl. CPC G01N 33/5005; G01N 21/6428; G01N 21/6456; G01N 2021/6439

(57) ABSTRACT

A cell information acquisition method of acquiring information on a cell by using autofluorescence emitted from the cell includes a metabolic status changing step of, by changing a metabolic status in the cell, changing at least one of a first autofluorescence luminance indicating a metabolic status of a first metabolic pathway and a second autofluorescence luminance indicating a metabolic status of a second metabolic pathway different from the first metabolic pathway, and an information acquisition step of, after the metabolic status changing step, acquiring information on the cell by using information on the first autofluorescence luminance and information on the second autofluorescence luminance.

### BACKGROUND

### Field of the Disclosure

The present disclosure relates to a cell information acquisition method of acquiring information on a cell by using autofluorescence emitted from the cell, and a program.

### Description of the Related Art

There is known a technology for acquiring information on a cell from an autofluorescence intensity of the cell. It has been reported that sugars that are nutrient sources are added to a solution containing microbes under test, and an autofluorescence intensity at that time is measured and compared with that of a sample without added sugars to make it possible to determine whether microbes under test are alive and have metabolic activities (Japanese Patent Laid-Open No. 2016-111940).

It has also been reported that microbial colonies inoculated on a solid or semi-solid culture medium are scanned and it is possible to perform detection, monitoring, and characterization of microbes in accordance with spectral information of autofluorescence (Japanese Patent Laid-Open No. 2016-073288).

However, in Japanese Patent Laid-Open No. 2016-111940, it is possible to detect microbes having metabolic activities by making a comparison with microbes having no metabolic activity, while, on the other hand, it is not possible to discriminate microbes having metabolic activities from each other. Microbes need to be fixed for measurement, so it is not possible to perform measurement before and after a test or progress during which a sample is continuously cultured. In Japanese Patent Laid-Open No. 2016-073288, a target is limited to an area on a solid or semi-solid culture medium, so the addition of a reagent needs to be performed at the time of creating the culture medium, and it is not possible to add or remove a reagent in process. It takes time to scan over a culture medium to measure one sample, so synchronicity in measurement among colonies is not guaranteed. Both Patent Literatures are intended to perform bulk measurement of a large number of samples, so the Patent Literatures are not appropriate for a method of measuring autofluorescence of each individual cell.

It has not been performed that a reagent is noninvasively added to a cell under test, color information derived from autofluorescence of each individual cell is acquired, information obtained from the color information is analyzed, and a metabolic status or cell type of each individual cell is not inferred from the analysis result.

### SUMMARY

When cells with different phenotypes are discriminated, information of live cells cannot be acquired through observation by staining, and there is an analysis using autofluorescence as noninvasive measurement; however, the metabolic status and activation status of a cell population are not uniform. For this reason, the inventors found that it was not easy to discriminate a cell type or determine a metabolic status by merely acquiring and analyzing autofluorescence at certain time for an object under test. In other words, the inventors found that there were a case where cells in which glucose metabolism is dominant are inactive, a case where cells in which respiration metabolism is dominant, and a case where those cannot be discriminated from each other by autofluorescence. The present disclosure provides cell information acquisition methods that acquire information on a cell by using autofluorescence emitted from the cell regardless of an activation status of the cell.

According to an embodiment of the present disclosure, a cell information acquisition method of acquiring information on a cell by using autofluorescence emitted from the cell includes a metabolic status changing step of, by changing a metabolic status in the cell, changing at least one of a first autofluorescence luminance indicating a metabolic status of a first metabolic pathway and a second autofluorescence luminance indicating a metabolic status of a second metabolic pathway different from the first metabolic pathway, and an information acquisition step of, after the metabolic status changing step, acquiring information on the cell by using information on the first autofluorescence luminance and information on the second autofluorescence luminance.

Further features of various embodiments of the present disclosure will become apparent from the following description of exemplary embodiments with reference to the attached drawings.

### BRIEF DESCRIPTION OF THE DRAWINGS

FIG. 1 is a flowchart that shows an example of an information acquisition method according to an embodiment of the present disclosure.

FIG. 2 is a diagram that shows the outline of an experiment related to an example of the present disclosure.

FIG. 3 shows a box-and-whisker plot that shows an analysis result of autofluorescence information under conditions that a reagent is added to COR-L23 cells or no reagent is added to COR-L23 cells and a box-and-whisker plot that shows an analysis result of autofluorescence information under conditions that a reagent is added to NCI-H322 cells or no reagent is added to NCI-H322 cells according to the example of the present disclosure.

FIG. 4 shows a box-and-whisker plot that shows an analysis result of mitochondria staining for confirming the effect of addition of a reagent on COR-L23 cells and a box-and-whisker plot that shows an analysis result of mitochondria staining for confirming the effect of addition of a reagent on NCI-H322 cells according to the example of the present disclosure.

FIG. 5 shows a box-and-whisker plot that shows an analysis result of lactic acid production amount measurement assay for a culture medium supernatant under conditions that a reagent is added to COR-L23 cells or no reagent is added to COR-L23 cells and a box-and-whisker plot that shows an analysis result of assay of measured amount of lactic acid produced in a culture medium supernatant under conditions that a reagent is added to NCI-H322 cells or no reagent is added to NCI-H322 cells according to the example of the present disclosure.

FIG. 6 shows autofluorescence images and slow muscle stained images under conditions that a reagent is added or no reagent is added, of skeletal muscles on which differentiation induction has been performed from C2C12 cells under respective conditions according to the example of the present disclosure.

FIG. 7 shows a box-and-whisker plot that shows an analysis result of autofluorescence image information of skeletal muscles to which differentiation induction has been performed from C2C12 cells by using an HS culture medium under conditions that a reagent is added or no reagent is added according to the example of the present disclosure.

FIG. 8 is a box-and-whisker plot that shows an analysis result of autofluorescence information of differentiation induced skeletal muscles from C2C12 cells by using an HS culture medium or an AIM-V culture medium under conditions that a reagent is added or no reagent is added according to the example of the present disclosure.

FIG. 9 is a scatter diagram that shows an analysis result of autofluorescence information of differentiation induced skeletal muscles from C2C12 cells by using an HS culture medium or an AIM-V culture medium under conditions that a reagent is added or no reagent is added according to the example of the present disclosure.

### DESCRIPTION OF THE EMBODIMENTS

A cell information acquisition method according to an embodiment of the present disclosure will be described by using a further specific configuration example; however, some embodiments of the present disclosure are not limited thereto.

A background art of the cell information acquisition method according to the present embodiment will be described. It has been already widely known that a plurality of materials that emit fluorescence is included in cell-intrinsic components. It is also known that, among these intrinsic fluorescent components, a plurality of coenzymes expresses information on a redox status or a metabolic status in a cell. HASEGAWA et al. particularly focused on coenzymes NADH and FAD of which the fluorescence luminance increases and decreases according to a redox status of a body tissue and developed a system configured to calculate a luminance ratio between the fluorescence wavelengths of the coenzymes from a photographed image of an organ and determine the redox status of the body tissue (K. Hasegawa, Y. Ogasawara, Transactions of the Visualization Society of Japan, 2010, 30, 11, 73-79).

A fluorescence lifetime imaging microscope has also been developed by a combination of a high-resolution microscope with femtosecond laser, and a change in autofluorescence wavelength component in process in which normal cells perform malignant transformation is measured from quantitative measurement of the absolute values of NADH and FAD in each individual cell is measured (Non-Patent Literature 2: M. C. Skala, K. M. Riching, A. Gendron-Fitzpatrick, J. Eickhoff, K. W. Eliceiri, J. G. White, and N. Ramanujam, PNAS, 2007, 104, 49, 19494-19499). In this way, measurement of a change in autofluorescence wavelength component is important for characterization of each individual cell.

However, generally, for cells of the same types, the absolute amount of coenzymes that are color developing factors of autofluorescence is not uniform, and the luminance and spectral information of autofluorescence constantly change. The luminance and spectral information of autofluorescence of each cell express information on cell type, redox status, and metabolic status. For example, there can be a case where it is not possible to discriminate, with autofluorescence, between a case where glucose metabolism of a cell in which glucose metabolism is intrinsically dominant is in an inactive state and a case where a glycolytic system of a cell in which respiration metabolism is dominant is active.

To solve this, in autofluorescence observation as a discrimination method on cell type or cell phenotype, when autofluorescence of each cell type is maximally increased and decreased by changing the metabolic status, it is conceivably possible to increase the probability of cell type separation by clarifying the intrinsic dominance of metabolism. In addition, it may be an important process for cell characterization to also get a change in autofluorescence, so there can be mostly desirable cases where live cells can be maintained even after the change.

In view of this, the inventors conceived that it was a task to acquire information on a cell by using autofluorescence emitted from the cell regardless of the activation status of the cell.

### Information Acquisition Method

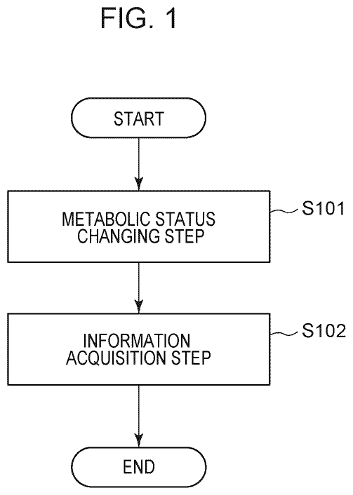

The cell information acquisition method according to the present embodiment will be described with reference to FIG. 1.

The cell information acquisition method according to the present embodiment acquires information on a cell by using autofluorescence emitted from the cell and includes at least the following steps.

(S101) A metabolic status changing step of, by changing a metabolic status in the cell, changing at least one of a first autofluorescence luminance indicating a metabolic status of a first metabolic pathway and a second autofluorescence luminance indicating a metabolic status of a second metabolic pathway different from the first metabolic pathway.

(S102) An information acquisition step of, after the metabolic status changing step, acquiring information on the cell by using information on the first autofluorescence luminance and information on the second autofluorescence luminance.

In this way, when the cell information acquisition method according to the present embodiment uses information on an autofluorescence luminance indicating a metabolic status after the metabolic status changing step, the cell information acquisition method is capable of acquiring information on the cell by using autofluorescence emitted from the cell regardless of the activation status of the cell.

### Information on Cell

The information on the cell according to the present embodiment is at least any one of information on a metabolic status of the cell, information on a type of the cell, and information on a phenotype of the cell.

### Information on Phenotype of Cell

In the present embodiment, the information on a phenotype of the cell may be information in which a group of the cells that are a target to acquire information is classified into two or more types. The information in which a group of the cells is classified into two or more types may be at least one selected from the group consisting of classification of live cell and dead cell, classification of cancer cell and non-cancer cell, classification of undifferentiated cell and differentiated cell, classification of differentiated cell and dedifferentiated cell, classification of cells in different differentiated states of the same-type differentiated cell type, and classification of epithelial cell and mesenchymal cell of the same-type cancer cell.

### Metabolic Status Changing Step

Examples of the metabolic status changing step according to the present embodiment include a step of adding a reagent to a cell culture system, a step of removing a reagent from a culture system, a step of changing a composition, such as pH, of a culture solution, a step of changing a culture system to a sealed system against outside air, and a step of changing a culture system from a sealed system to an open state; however, the configuration is not limited thereto.

In the cell information acquisition method according to the present embodiment, to be applied to an alive cell and then make it possible to continuously culture the cell, the metabolic status changing step can be noninvasive to the cell; however, the metabolic status changing step may be invasive.

In the metabolic status changing step according to the present embodiment, the first metabolic pathway and the second metabolic pathway each may be any one of a glycolytic system, a TCA cycle, and an electron transfer system.

The metabolic status changing step may include a step of changing at least any one of a metabolic status of the first metabolic pathway and a metabolic status of the second metabolic pathway. The metabolic status changing step may include, for example, a step of accelerating metabolism of the first metabolic pathway and inhibiting metabolism of the second metabolic pathway.

Specifically, the metabolic status changing step may include an addition step of adding a reagent having a component that changes at least any one of a metabolic status of the first metabolic pathway and a metabolic status of the second metabolic pathway. The addition step may include, for example, a step of adding a reagent having a component that accelerates metabolism of the first metabolic pathway and that inhibits metabolism of the second metabolic pathway.

The metabolic status changing step according to the present embodiment may be a step capable of reversibly changing a metabolic status to a metabolic status before the metabolic status changing step.

### Noninvasive

In the present embodiment, a noninvasive technique does not intentionally and immediately perform treatment that has the effect of killing cells and means that it is possible to try continuously culturing by, after treatment, replacing the status of a culture medium with the status of the culture medium before a reagent is added, or another status. A noninvasive technique means, for example, that a process on the metabolic status changing step or treatment having the effect of killing cells for autofluorescence measurement is not performed, for example, formalin fixation is not performed at the time of measurement; however, the noninvasive technique is not limited thereto.

### Reagent

A metabolism changing reagent made of any one of a metabolism enhancer, a metabolism inhibitor, and a metabolism maintenance agent to each metabolic pathway of the cell or a combination of them may be used as a reagent for changing a metabolic status according to the present embodiment. The reagent according to the present embodiment may include, for example, at least one selected from the group consisting of a glycolytic system accelerator, a glycolytic system inhibitor, a TCA cycle accelerator, a TCA cycle inhibitor, an electron transfer system accelerator, and an electron transfer system inhibitor. Examples of the reagent according to the present embodiment include D-glucose and antimycin A, 2-deoxy-D-glucose, WZB117, pyruvate, UK 5099, rotenone, malonate, Atpenin A5, oligomycin, azide salt, carbon dioxide gas, oxygen gas, CCCP, and FCCP. A reagent containing D-glucose and antimycin A can be used as the reagent according to the present embodiment. The reagent according to the present embodiment can be noninvasive to the cell that is a target to acquire information. When the reagent is removed, the reagent may allow a metabolic status to reversibly change to a metabolic status before the reagent is added to the cell.

### Other Steps

In the present embodiment, the cell information acquisition method may further include a step of, before the metabolic status changing step, acquiring information on the cell in a state where metabolism of the cell remains unchanged. The cell information acquisition method may further include, after the information acquisition step, a classification step of classifying one or more pieces of data in the information acquired from the autofluorescence information into two or more clusters.

The cell information acquisition method may further include a step of culturing the cell after the information acquisition step.

### Program

A program according to the present embodiment may be a program for causing a computer to execute the cell information acquisition method.

### Cell

In the present embodiment, the cell that is a target to acquire information is not limited, and a mammalian cell can be used. Examples of the target cell include a culture cell, a cell of a tissue section, and a cell extracted from a tissue or a living sample. Examples of the target cell include a normal cell, a cancer cell, a mixed culture of a cancer cell and a normal cell, a mixed culture of cancer cells, a mixed culture of normal cells, and a cell included in a mixed tissue or blood. The target cell is a cell in which the presence or absence of the characters of allogenic cells, such as a slow muscle fiber and a fast muscle fiber of the same-type differentiation cell type, epithelial-mesenchymal transformation of a cancer cell, differentiation induction from an undifferentiated cell, and cell senescence, is estimated.

In the method according to the present embodiment, autofluorescence information or autofluorescence information and cell morphologic information is acquired from a cell.

A cell may be in any state as long as it is possible to acquire information of the cell.

For example, when an image acquisition apparatus is a general microscope, a cell may be in a state suspended, adhering, or cultured in a petri dish, a plate, a flask, or the like or may be contained in a section. When image capture is possible, a cell may be the one present in a living body, such as a blood and a tissue.

The method according to the present embodiment has such a feature that the method can be applied to a live cell. A target is not limited to a live cell and may be, for example, a sample in which a live cell and a dead cell are mixed.

### Autofluorescence

Autofluorescence means fluorescence that a material included in a cell or a tissue emits upon receiving application of an excitation wavelength. Particularly, examples of an intrinsic component that emits autofluorescence include autofluorescence molecules produced in a cell, such as NAD(P)H, flavines (such as FAD), collagen, fibronectin, tryptophan, and folic acid.

In the method according to the present embodiment, it is not necessary to identify what the intrinsic component emitting autofluorescence is, and autofluorescence includes fluorescence that an intrinsic component other than the fluorescent intrinsic component, of which examples have been described above, emits.

One or more pieces of autofluorescence information may be acquired from one sample including a plurality of cells. Autofluorescence information is obtained from fluorescence that is emitted as a result of applying excitation light with a specific wavelength. Excitation light may be light with one wavelength or may be light made up of rays of light with a plurality of wavelengths. Fluorescence may be light with one wavelength or may be light made up of rays of light with a plurality of wavelengths.

An examples of application of excitation light is application of excitation light through a short pass filter that cuts fluorescence longer than or equal to a fluorescence wavelength range intended to be measured, with the use of a white light source, such as a mercury lamp and a xenon lamp, that emits light with a wavelength of longer than or equal to 350 nm. Another example is application of excitation light through a short pass filter that cuts fluorescence longer than or equal to a fluorescence wavelength range intended to be measured, with the use of an LED that emits light in a specific wavelength range having a center wavelength as a light source; however, application of excitation light is not limited thereto.

Examples of the wavelength at the time of acquiring autofluorescence information include 365 nm excitation/430 nm long pass filter observation, 365 nm excitation/530 nm long pass filter observation, 395 nm excitation/430 nm long pass filter observation, 395 nm excitation/530 nm long pass filter observation, and 470 nm excitation/530 nm long pass filter observation; however, the wavelength is not limited thereto. Here, 365 nm excitation/430 nm long pass filter observation means to observe autofluorescence produced as a result of excitation using light with a wavelength of 365 nm, with a 430 nm long pass filter. The same applies to the cases of the other wavelengths.

### Metabolism

Metabolism is a chemical reaction that accompanies a series of ATP syntheses that an organism performs by using inorganic materials and organic compounds, taken in from the outside, as raw materials, and the flow of the series is referred to as a metabolic pathway.

Examples of the metabolic pathway in the method according to the present embodiment include a glycolytic system, a TCA cycle, and an electron transfer system; however, the metabolic pathway is not limited thereto.

The metabolic status of the cell according to the present embodiment means a comprehensive status of an active state or an inactive state of each metabolic pathway in each individual cell.

### Autofluorescence Information

Autofluorescence information includes the luminance of autofluorescence and may further include positional information, area information, and other information, of luminance. Luminance is not limited as long as the luminance is a value indicating a fluorescence intensity, and any index may be used. Luminance may be replaced with intensity.

Luminance may be indicated on a color space coordinate system and may be a value obtained from the coordinate system. An RGB color space may be used as an example of the color space. Luminance may be a value calculated as an index of at least any one of R, G, and B in the RGB color space. Luminance may be a value calculated as an index in a Lab color space, an HSV color space, or an HLS color space. Luminance information may be described by using a color space other than these.

Alternatively, luminance may be described by using spectral information of autofluorescence.

In the specification, in a color space, R may have a wavelength of longer than or equal to 600 nm and shorter than or equal to 800 nm, G may have a wavelength of longer than or equal to 500 nm and shorter than 600 nm, and B may have a wavelength of longer than or equal to 350 nm and shorter than 500 nm.

As autofluorescence information of each individual cell, one or more pieces of autofluorescence information can be determined for each cell, and, typically, one piece autofluorescence information is determined for each cell.

Autofluorescence information of each individual cell may be luminance of autofluorescence of each individual cell and, furthermore, a representative value of luminance of the cell may be autofluorescence information of that cell. A minimum value, a maximum value, an average value, a median value, or the like may be used as a representative value.

For example, acquiring autofluorescence information by using R in a color space as an index means acquiring the value of luminance with any one wavelength in a range longer than or equal to 600 nm and shorter than or equal to 800 nm for each individual cell in an autofluorescence image. To classify a plurality of cells into two or more groups, the value of luminance for at least one index of R, G, and B can be acquired.

### Information Acquisition Step

The information acquisition step according to the present embodiment acquires at least any one of information on a metabolic status of the cell and information on a cell type of the cell by using information on the first autofluorescence intensity and the second autofluorescence intensity.

In the information acquisition step according to the present embodiment, an apparatus that acquires these pieces of information may be any apparatus capable of comprehensively obtaining those pieces of information. The apparatus may be, for example, an image acquisition apparatus, such as a camera, that captures and acquires an image via an optical system including a magnifier, such as a microscope, or may be a flow cytometer or the like.

The information acquisition step according to the present embodiment has such a feature that the information acquisition step can be applied to a live cell and, after the application, the cell can be continuously cultured, so the information acquisition step can be noninvasive to the cell and may be invasive.

In the present embodiment, since the reagent addition step and the autofluorescence acquisition step are noninvasive, at least one of the reagent addition step and the autofluorescence acquisition step may be repeated one or more times for the sample. Thus, it is possible to acquire cell information in a plurality of metabolic statuses for one sample. For example, for a cell in a prepared culture, the autofluorescence acquisition step may be performed before addition of a reagent, the reagent addition step may be performed, and then the autofluorescence acquisition step may be performed. Alternatively, the reagent addition step may be performed, the autofluorescence acquisition step may be performed, and then the autofluorescence acquisition step may be performed with a culture solution to which no reagent is added. In other words, for the same sample, the addition step of a reagent (1) may be performed, then the autofluorescence acquisition step may be performed, furthermore, the addition step of another reagent (2) may be performed, and then the autofluorescence acquisition step may be performed.

The cell information acquisition method may include a step of adding the reagents different from each other to respectively a plurality of samples each including the cell and acquiring an autofluorescence luminance after the addition.

### Morphologic Information

The cell information acquisition method may include a step of acquiring morphologic information separately from the autofluorescence information.

Morphologic information includes the position, size, area, shape, and thickness of each individual cell, a distribution of the cells, and the like. A region of each individual cell can be acquired by using the morphologic information. Morphologic information, for example, may be acquired from a bright field image, an autofluorescence image, a stained image, or the like of cells with an image acquisition apparatus and may be acquired from forward scattered light or side scattered light with flow cytometry.

Morphologic information may be, for example, information on an autofluorescence image or information on a masked image used so as to be overlaid on autofluorescence information. The autofluorescence image and the masked image are created from the autofluorescence image.

A masked image functions to extract information corresponding to each individual cell of the autofluorescence information and mask the other information.

### Region Extraction Image

A region extraction image is an image that is acquired to obtain morphologic information of each individual cell in an autofluorescence image and can be the one with which each individual cell itself, cell membrane, nucleus, and the other cell organelles can be identified. A region extraction image can be a bright field image or a phase difference image and may be acquired as, for example, a bright field image or a fluorescence image obtained after specific cell organelles that are nuclei as an example are stained. However, when the region extraction image is a fluorescence image, the excitation wavelength or fluorescence wavelength can be selected so as not to overlap those wavelengths of autofluorescence. One or more region extraction images can be acquired from one sample.

A region extraction image may be a bright field image obtained by enlarged image formation and photographing in a bright field. Alternatively, a region extraction image may be a fluorescence photographed image of a stained sample or a non-stained sample.

### Third Information

The cell information acquisition method may include a step of acquiring third information separately from the autofluorescence information or the morphologic information. Third information may be information acquired from a bright field image, may be information acquired from a fluorescence image, or may be information acquired from other than images. Examples of the third information include information on whether there is a specific marker in a cell and other information and include an immunostaining image in which a desired marker is specifically stained.

### Bright Field Image

A bright field image can be used to acquire morphologic information or third information. A bright field image acquisition method is, for example, as follows. A light source with a visible light wavelength or a white light source that is a mixture of a plurality of light source wavelengths is applied parallel to an optical axis of an objective lens or not parallel but at an angle not perpendicular to the optical axis. Then, reflected light or diffraction light due to birefringence from a target sample and their interference can be detected.

### Fluorescence Image

A fluorescence image may be used for autofluorescence information, morphologic information, or third information. An example of a fluorescence image acquisition method will be described below. A light source with a center wavelength set to a specific wavelength in a range from ultraviolet to visible light as a fluorescence excitation light source wavelength is applied to a sample parallel to the optical axis of the objective lens or at an angle at least not perpendicular to the optical axis to excite fluorescence of the sample. Fluorescence produced by excitation is detected via a cut filter installed in front of or behind an observation-side objective lens or the like. As an excitation light and fluorescence observation cut filter, a wavelength cut filter is selected such that part of an excitation light source wavelength does not pass through the observation-side fluorescence cut filter.

### Visualization Step

In the present embodiment, it is possible to visualize the result of information obtained so that a user easily understands the result of the information. For example, the result of information obtained can be visualized as a one-dimensional, two-dimensional, or three-dimensional graph with at least one axis set for the luminance of autofluorescence of each individual cell. The graph may be a one-dimensional graph with an axis set for only the luminance of autofluorescence. Alternatively, the graph may be a two-dimensional or three-dimensional graph with axes set for two or more luminances of autofluorescence. Furthermore, the graph may be a two-dimensional or three-dimensional graph with an axis set for the luminance of autofluorescence and another axis set for morphologic information or third information, other than the luminance of autofluorescence. A plurality of cells can be visually classified into two or more groups by using a graph.

A graph can be anything that visualizes numeric information. A graph just needs to visualize information. A histogram may be illustrated for a one-dimensional graph, and a scatter diagram may be illustrated for a two-dimensional or three dimensional graph; however, the graph is not limited thereto. Furthermore, a probability density function plot of kernel density estimation or the like, a heat map, or the like may be used as a graph.

### Cell Status Inference Step

In a cell status inference step, a plurality of cells can be classified into two or more groups by using autofluorescence information, morphologic information, or third information of each individual cell. For example, a plurality of cells can be classified into two or more groups by unsupervised machine learning.

Examples of the unsupervised machine learning include principal component analysis, k-means methods, and GMM.

The two or more classified groups each may be associated with a phenotype of cells group by group. Examples of a combination of phenotypes respectively associated with two or more groups include a combination of live cell and dead cell, a combination of cancer cell and non-cancer cell, a combination of cancer cell groups respectively having different malignancies, a combination of undifferentiated cell and differentiated cell, a combination of differentiated cell and dedifferentiated cell, a combination of slow muscle fiber and fast muscle fiber of the same-type differentiated cell type, and a combination of epithelial cell and mesenchymal cell of the same-type cancer cell.

Therefore, the present embodiment may be used to, for example, determine the malignancy of cancer cell in a tissue or a culture cell, maintain and culture undifferentiated cells of various stem cells, and determine a difference in differentiation in differentiation induction or the like.

### Acquisition of Quantitative Information

In the present embodiment, the cell information acquisition method may include a step of acquiring quantitative information on the groups classified by the classification step. Here, quantitative information includes the number of cells that belong to each group, the ratio of the numbers of cells that belong to each group, the ratio of at least two luminances of RGB in a color space, and the ratio of representative values of luminances in each group. Here, a minimum value, a maximum value, an average value, a median value, or the like may be used as a representative value.

### EXAMPLE

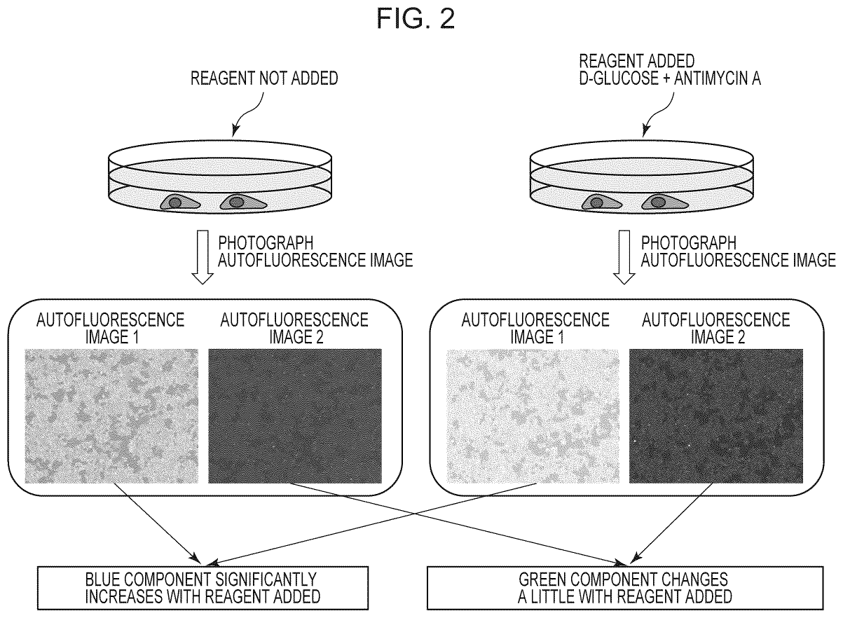

Hereinafter, an embodiment of the present disclosure will be described in further details by using an example; however, some embodiments of the present disclosure are not limited to the following configuration. FIG. 2 is a diagram that shows the outline of an actual experiment according to the present example. In FIG. 2, two sheets of NCI-H322 cells cultured with a similar culture method were prepared, and a sample with a reagent added and a sample with antimycin A and D-glucose added were created. A B component of a cell autofluorescence image (autofluorescence image (1)) of 365 nm excitation/430 nm long pass filter observation and a G component of the cell autofluorescence image (autofluorescence image (2)) of 365 nm excitation/430 nm long pass filter observation were acquired.

### Experiment Using Human Lung Cancer Cells

### Culture of Cells

Human lung cancer cells NCI-H322 (KAC Co., Ltd.) were cultured in a culture medium 103 for cell culture (KAC Co., Ltd.). Human lung cancer cells COR-L23 (KAC Co., Ltd.) were cultured in a culture medium 103 for cell culture (KAC Co., Ltd.). A cell culture vessel was a 35 mm glass bottom dish (IWAKI) for any case.

### Preparation of Adding Metabolism Changing Reagent (Removal of D-Glucose))

A culture medium in which 10% of Fetal bovine serum (hereinafter, FBS, produced by NACALAI TESQUE, INC.) and 1% of penicillin-streptomycin solution (produced by FUJIFILM Wako Pure Chemical Corporation) were added to RPMI-1640 culture medium (produced by FUJIFILM Wako Pure Chemical Corporation) not containing D-glucose was prepared. The cell culture solution was removed from NCI-H322 cells or COR-L23 cells, the culture medium prepared as described above was added, and then the cells were incubated for 30 minutes at 37° C. in 5% CO₂.

### Addition of Metabolism Changing Reagent

After D-glucose was removed, a sample in which only a D-glucose solution (final concentration 4500 mg/mL, Tokyo Chemical Industry Co., Ltd.) was added and a sample in which a D-glucose solution and antimycin A derived from actinomycetes (hereinafter, antimycin A, final concentration 1 μM, Sigma-Aldrich) were added were prepared and were incubated on the culture medium for an hour at 37° C. in 5% CO₂.

### Autofluorescence Image Observation

The cell culture solution was replaced with Phosphate buffer saline (hereinafter, PBS, produced by Thermo Fisher), an autofluorescence image captured through a 430 nm long pass filter by applying excitation light to a 365 nm LED (produced by Asahi Spectra Co., Ltd.) light source via a 400 nm short pass filter to excite the light source, and an autofluorescence image captured through a 530 nm long pass filter by applying excitation light to a 450 nm LED (produced by Asahi Spectra Co., Ltd.) light source via a 470 nm short pass filter to excite the light source were obtained.

### Mitochondria Staining, Nuclear Staining

A membrane potential difference between nucleus and mitochondria was visualized by staining mitochondria with MitoPT™—TMRE assay kit (produced by Immunochemistry Technologies) and staining the nucleus with Hoechst dye (produced by Dojindo Laboratories) for culture cells.

### Measurement of Amount of Lactic Acid Produced

A culture medium supernatant was collected from the culture cells, and the amount of lactic acid in the supernatant was measured by Lactate Assay Kit-WST (produced by Dojindo Laboratories).

### Stained Image Observation

For nuclei, a fluorescence image of nuclei captured through a 430 nm to 490 nm band-pass filter by applying excitation light to a 365 nm LED (produced by Asahi Spectra Co., Ltd.) light source via a 400 nm short pass filter to excite the light source was obtained. For a mitochondria membrane potential difference, a mitochondria stained image captured through a 570 nm to 630 nm band-pass filter by applying excitation light to a 530 nm LED (produced by Asahi Spectra Co., Ltd.) light source via a 550 nm short pass filter to excite the light source was obtained.

### Extraction of Autofluorescence Information of Cells

The area of a nucleus region was acquired and labelled. Each autofluorescence image was merged with the nucleus regions, and an autofluorescence component of the nucleus region was acquired for each individual cell. RGB components of each of the autofluorescence images were separated and the R component, G component, and B component of autofluorescence of the nucleus region were acquired for each individual cell. Detailed data (nucleus areas, a minimum value, a maximum value, an average value, and a median value among the nucleus areas, and the like) were output for each labelled cell.

### Box-and-Whisker Plot of Autofluorescence Luminance Information

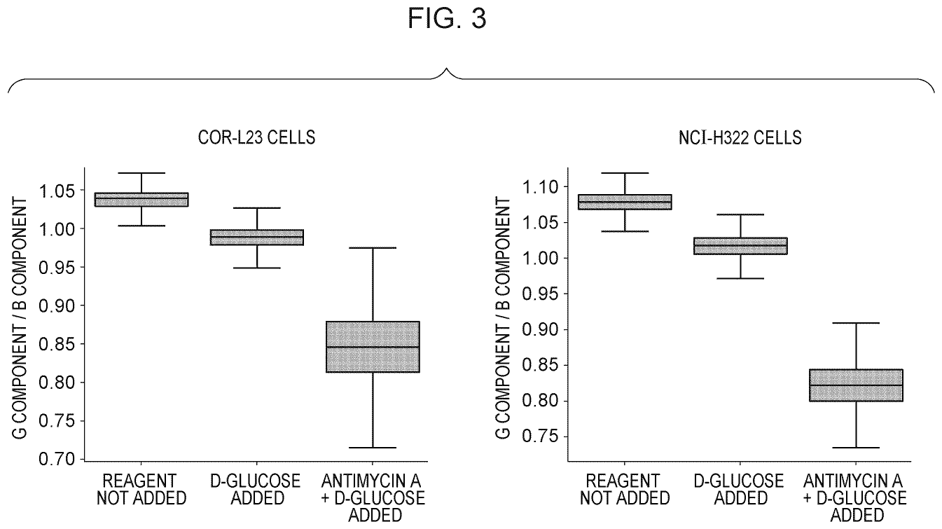

The luminance information of the cells was used to obtain an average value of B component of the autofluorescence image of 365 nm excitation/430 nm long pass filter observation and an average value of G component of the autofluorescence image of 365 nm excitation/430 nm long pass filter observation. Average value of G component/Average value of B component was calculated cell by cell and plotted as a box-and-whisker plot (FIG. 3).

The ordinate axis represents a ratio between an average value of G component of an autofluorescence image in the RGB color space of 365 nm excitation/430 nm long pass filter observation in each of the cells and an average value of B component of the autofluorescence image in the RGB color space of 365 nm excitation/430 nm long pass filter observation in a corresponding one of the cells.

### Extraction of Luminance Information of Mitochondria Stained Image, and Plot

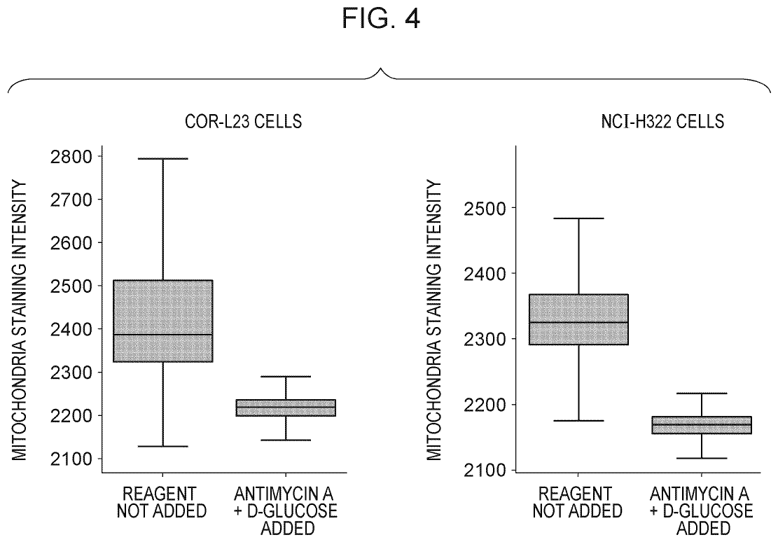

A method according to an embodiment of the present disclosure was applied to not an autofluorescence image but a stained image as in the case of the method applied to an autofluorescence image. In other words, a luminance value of each individual cell in a stained image was recorded, and the area of a nucleus region was acquired by image processing. For a mitochondria stained image, color information of the nucleus region was acquired for each individual cell. As in the case of extraction of luminance information of an autofluorescence image, positional data of the cells and detailed data (nucleus areas, and a minimum value, a maximum value, an average value, and a median value among the nucleus areas) of the cells were recorded in correspondence with each other. Then, mitochondria luminance values were plotted with the following ordinate axis and abscissa axis (FIG. 4).

The ordinate axis represents an average value of R component in the RGB color space of 530 nm excitation/570 nm to 630 nm band-pass filter observation in each cell.

### Plot of Measurement Result of Amount of Lactic Acid Produced

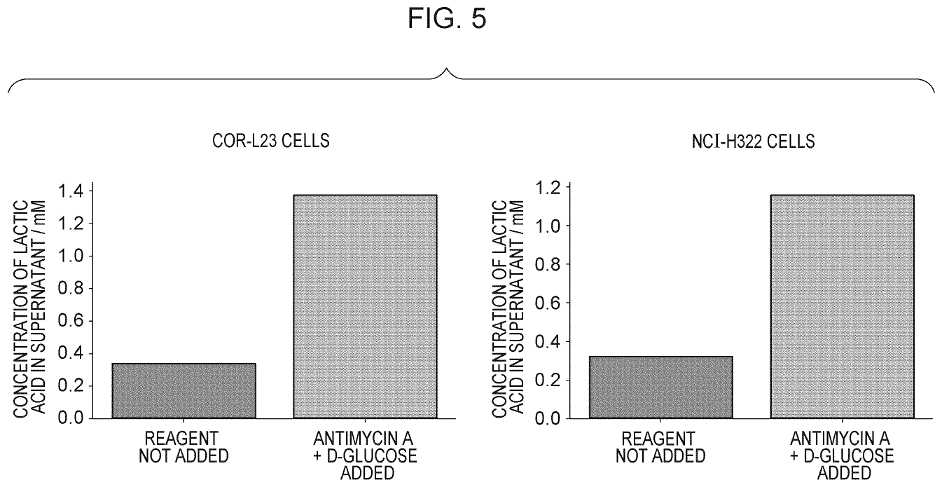

A culture medium supernatant was collected from the culture cells, the amount of lactic acid in the supernatant was measured by Lactate Assay Kit-WST (produced by Dojindo Laboratories), and the measurement result was plotted with the ordinate axis representing the concentration of lactic acid in the supernatant (FIG. 5).

### Experiment Using Mouse Myoblast

### Culture of Cells

Mouse myoblast cell strain C2C12 (KAC Co., Ltd.) was cultured in a cell maintenance medium (KAC Co., Ltd.). A 35 mmφ glass bottom dish (Matsunami Glass Ind., Ltd.) was used as a cell culture vessel.

### Differentiation Induction

As a general skeletal muscle differentiation induction, at the stage where a confluence rate becomes higher than or equal to 80% to 90%, a culture solution was put into a culture medium (hereinafter, HS culture medium) in which 2% of Horse serum (hereinafter, HS, produced by Cosmo Bio Co., Ltd.) and 1% of penicillin-streptomycin solution (produced by FUJIFILM Wako Pure Chemical Corporation) were added to DMEM culture medium (produced by FUJIFILM Wako Pure Chemical Corporation), and differentiation induction to a skeletal muscle was started. After 48 hours, the culture medium was replaced with a new culture medium of the same type again and further cultured for 48 hours to perform differentiation induction culture. In addition, differentiation induction using a differentiation induction method in which the HS culture medium was changed to AIM-V™ culture medium (produced by Thermo Fisher) (hereinafter, AIM-V culture medium) and of which the appearance ratio of slow muscle was higher than that of the above-described general differentiation induction (M. Jang, J. Scheffold, L. M. Rost, H. Cheon, P. Bruheim, Sci Rep. 2022, 12, 827). The conditions of start of differentiation induction and culture medium replacement are exactly the same as the above-described conditions.

### Preparation of Adding Metabolism Changing Reagent (Removal of D-Glucose)

A culture medium in which 1% of penicillin-streptomycin solution (produced by FUJIFILM Wako Pure Chemical Corporation) was added to DMEM culture medium (produced by FUJIFILM Wako Pure Chemical Corporation) not containing D-glucose was prepared. The cell culture solution was removed from C2C12 cells in culture, the culture medium prepared was added, and then the cells were incubated for 30 minutes at 37° C. in 5% CO₂.

### Addition of Metabolism Changing Reagent

After D-glucose was removed, a D-glucose solution (final concentration 4500 mg/mL, Tokyo Chemical Industry Co., Ltd.) and antimycin A (final concentration 5 μM, Sigma-Aldrich) were added to a culture medium that had been incubated for 30 minutes at 37° C. in 5% CO₂.

### Autofluorescence Image Observation

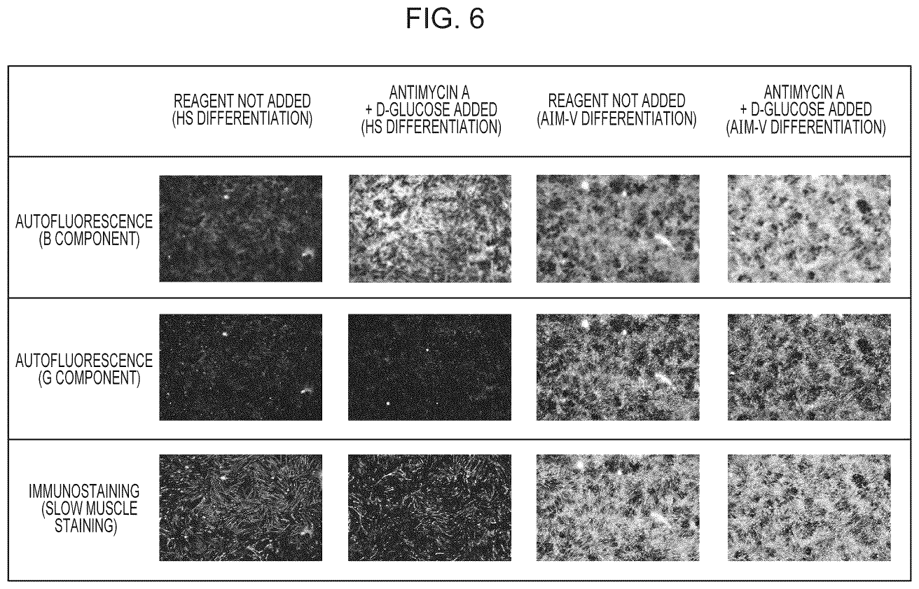

The cell culture solution was replaced with Phosphate buffer saline (hereinafter, PBS, produced by FUJIFILM Wako Pure Chemical Corporation), an autofluorescence image captured through a 430 nm long pass filter by applying excitation light to a 365 nm LED (produced by Asahi Spectra Co., Ltd.) light source via a 400 nm short pass filter to excite the light source was obtained as a B component, and an autofluorescence image captured through a 530 nm long pass filter by applying excitation light to a 395 nm LED (produced by Asahi Spectra Co., Ltd.) light source via a 405 nm short pass filter to excite the light source was obtained as a G component (the top row and the middle row in FIG. 6).

### Staining of Various Muscle Fiber Types, and Nuclear Staining

For culture cells, a slow muscle (type I fibers) Anti-Slow Skeletal Myosin Heavy chain (ab11083, produced by abcam) and its corresponding secondary antibody Anti IgG (H+L), Rabbit (Donkey) CF™ 488A (produced by Biotium), a fast muscle (type II fibers) Anti-Fast Skeletal Myosin Heavy chain (ab91506, produced by abcam) and its corresponding secondary antibody Anti IgG(H+L), Rabbit (Donkey) CF™ 555 (produced by Biotium), and nuclei were stained with Hoechst dye (produced by Dojindo Laboratories) to visualize the slow muscle, the fast muscle, and the nuclei.

### Stained Image Observation

As for slow muscle staining, a slow muscle stained image captured through a 520 nm to 540 nm band-pass filter by applying excitation light to a 470 nm LED (produced by Asahi Spectra Co., Ltd.) light source via a 490 nm short pass filter to excite the light source was obtained (the bottom row in FIG. 6). As for fast muscle staining, a fast muscle stained image captured through a 570 nm to 630 nm band-pass filter by applying excitation light to a 530 nm LED (produced by Asahi Spectra Co., Ltd.) light source via a 550 nm short pass filter to excite the light source was obtained. For nuclei, a fluorescence image of nuclei captured through a 430 nm to 490 nm band-pass filter by applying excitation light to a 365 nm LED (produced by Asahi Spectra Co., Ltd.) light source via a 400 nm short pass filter to excite the light source was obtained.

### Extraction of Autofluorescence Information of Cells

Regions having differentiated into skeletal muscles were extracted, and an autofluorescence component of each skeletal muscle region was acquired. RGB components of each of the autofluorescence images were separated and the R component, G component, and B component of autofluorescence of each skeletal muscle region were acquired. The results of each labelled skeletal muscle region were output.

### Box-and-Whisker Plot of Autofluorescence Luminance Information

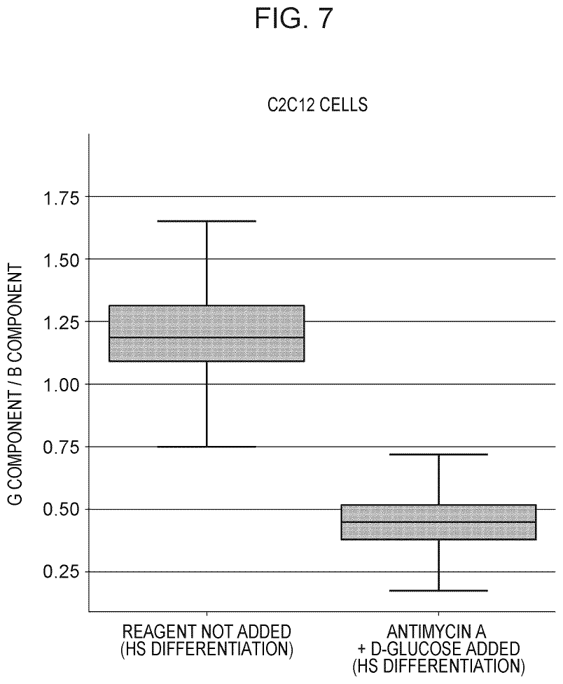

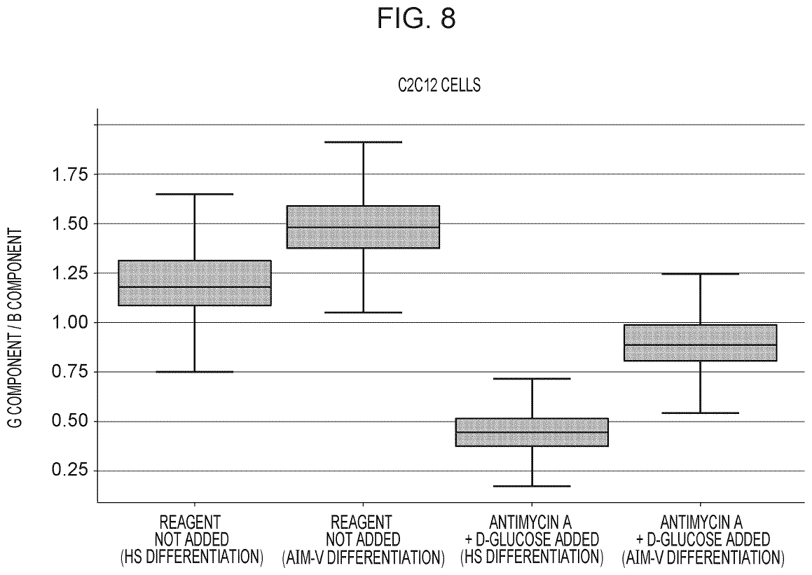

The luminance information of the skeletal muscle regions was used to obtain an average value of a B component of the autofluorescence image of 365 nm excitation/430 nm long pass filter observation and an average value of a G component of the autofluorescence image of 395 nm excitation/530 nm long pass filter observation. Average value of the G component/Average value of the B component was calculated for each skeletal muscle region and plotted as a box-and-whisker plot (FIGS. 7 and 8).

The ordinate axis represents a ratio between a G component of an autofluorescence image in the RGB color space of 395 nm excitation/530 nm long pass filter observation in each skeletal muscle region and a B component of the autofluorescence image in the RGB color space of 365 nm excitation/430 nm long pass filter observation in a corresponding one of the skeletal muscle regions.

### Scatter Diagram Plot of Autofluorescence Luminance Information

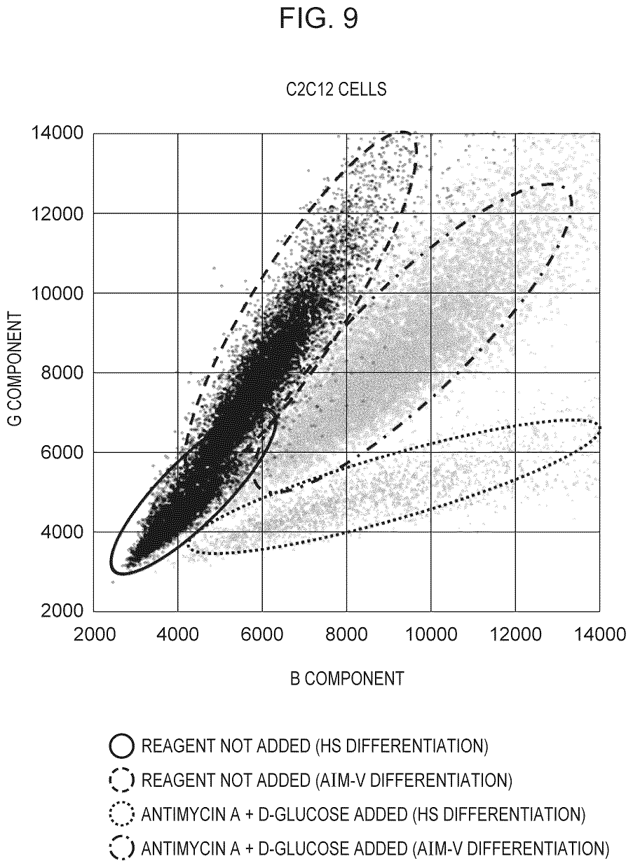

An average value of the B component and an average value of the G component were calculated for each skeletal muscle region and plotted as a scatter diagram (FIG. 9).

The abscissa axis represents a B component of an autofluorescence image in the RGB color space of 365 nm excitation/430 nm long pass filter observation in each skeletal muscle region, and the ordinate axis represents a G component of the autofluorescence image in the RGB color space of 395 nm excitation/530 nm long pass filter observation in a corresponding one of the skeletal muscle regions.

Hereinafter, experiments will be described. Preparation and the like of reagents have been described above, so the description thereof is omitted here.

### Box-and-Whisker Plot (FIG. 3) of Luminance Values of Autofluorescence of NCI-H322 Cells and COR-L23 Cells

NCI-H322 cells were cultured in a 35 mm glass bottom dish, and COR-L23 cells were cultured in a 35 mm glass bottom dish. Samples were created by adding a metabolism changing reagent (only D-glucose or antimycin A+D-glucose) to NCI-H322 cells and COR-L23 cells.

As negative control, only removal of D-glucose was performed for both NCI-H322 cells and COR-L23 cells to create samples not added with a metabolism changing reagent. An autofluorescence image of each of the samples was captured. Subsequently, nuclear staining and mitochondria staining were performed to stain nuclei and mitochondria, and similarly stained images were captured with an image capturing apparatus. Autofluorescence luminances of cells in a nucleus region of each cell were acquired by using these images separately by excitation and observation wavelength and RGB, and the luminances in each individual cell region were averaged to get the luminance value of the cell.

For the obtained autofluorescence luminance values of the cells, a box-and-whisker plot was drawn with the ordinate axis representing a ratio between a G component of an autofluorescence image of 365 nm excitation/430 nm long pass filter observation and a B component of the autofluorescence image of 365 nm excitation/430 nm long pass filter observation in each cell (FIG. 3).

There is no significant difference in value between the D-glucose added samples and the negative control samples; whereas there is a significant difference in G component/B component between the antimycin A+D-glucose added samples and the negative control samples. This demonstrates that autofluorescence changes as a result of addition of antimycin A+D-glucose and the change can be quantitatively measured.

As described above, autofluorescence emitted from cells is due to fluorescence of coenzymes (NADH, FAD, and the like) related to metabolism, and the proportion of appearance of activated coenzymes varies depending on a difference in the degree of metabolic pathway (glycolytic system, TCA cycle, electron transfer system, and the like) appearing in each cell. Those activated coenzymes have molecules that emit fluorescence, and the fluorescence wavelength of each of the coenzymes varies, so color of autofluorescence varies among cells having different metabolic pathways. For this reason, when there is a plurality of cell types having different metabolic pathways in one field of view, the cell types can be discriminated in accordance with a difference in the color of autofluorescence. However, for a cell population, the metabolic status is not in the certain same state but is constantly varying. For this reason, for a cell population under test, the color of autofluorescence obtained at one time has a distribution, and a distribution of the color of autofluorescence can overlap if cells of different types are mixed, so discrimination is not easy. However, cells in different metabolic statuses differ in response to a reagent, so it is presumable that a difference in distribution can be remarkable and discrimination becomes easy by observing and analyzing a change in autofluorescence when a reagent is added. For example, in this example, respiratory metabolism also increases with activation of glycolytic system with addition of only D-glucose, so the ratio between a blue component and a green component of autofluorescence wavelengths due to these factors does not significantly change as compared to before addition.

Antimycin A is known as an inhibitor for the electron transfer system, while it has also been reported as a reagent that activates the glycolytic system. For this reason, when addition of D-glucose that is a raw material of the glycolytic system is performed together, it is expected to further activate only the glycolytic system as compared to addition of antimycin A alone.

At this time, acceleration of NADH amount occurs as a result of activation of the glycolytic system. As a result, it is presumable that, for autofluorescence intensities of cells, only a blue component that has a fluorescence wavelength of NADH mainly increases. On the other hand, the amount of produced pyruvic acid that is a product of glycolytic system and also a raw material of TCA cycle increases as a result of activation of the glycolytic system; however, since the electron transfer system is inhibited, there is no reason why FAD increases equivalently to a glycolytic system reaction unlike a normal metabolic status. The TCA cycle is a reaction path of which a reaction rate is relatively lower than that of the glycolytic system. For this reason, it is expected that, as a result of addition of antimycin A or glucose, the degree of activation on the TCA cycle is lower than the degree of activation on the glycolytic system. As a result, it is presumable that a change in metabolic status caused by activation of TCA cycle caused by those reagents is acceleration of FAD and the accompanied increase in green component relatively gently increases, remains unchanged, or reduces as compared to an increase in blue component due to NADH acceleration. As a result, the result of FIG. 3 is presumably obtained because the ratio of G component/B component has increased as a result of addition of a reagent.

### Box-and-Whisker Plot (FIG. 4) of Luminance Values of Mitochondria Stained NCI-H322 Cells and COR-L23 Cells

From mitochondria stained images and mitochondria stained luminance value data of each cell, acquired from the mitochondria stained images, a box-and-whisker plot (FIG. 4) was drawn with the ordinate axis representing an average value of R component in the RGB color space of 530 nm excitation/570 nm to 630 nm band-pass filter observation in each cell.

Since there is a significant difference in R component between the antimycin A+D-glucose added samples and the negative control samples, it is expected that the activation state of mitochondria changes as a result of addition of antimycin A+D-glucose.

A mitochondria staining reagent is stained according to a mitochondria membrane potential difference to reflect a metabolic status in mitochondria. When mitochondria are in an active metabolic status and as a potential difference between inside and outside increases, the mitochondria are intensively stained by staining reagent.

As described above, antimycin A is an inhibitor for the electron transfer system and inhibits transport of protons between the inside and outside of mitochondria membrane. For this reason, it is expected that addition of antimycin A leads to weak staining in relation to mitochondria staining. Actually, the antimycin A+D-glucose added samples lead to the result of FIG. 4 in which the luminance of mitochondria stained result has decreased as compared to negative control. This demonstrates that the activation state of mitochondria actually decreases as a result of addition of antimycin A.

### Plot (FIG. 5) of Amount of Lactic Acid Produced in NCI-H322 Cells and COR-L23 Cells

Cells added with antimycin A+D-glucose were additionally prepared, and the amount of lactic acid produced was measured with negative control. A plot (FIG. 5) was drawn with the ordinate axis representing the concentration of lactic acid in the culture medium supernatant.

From the result, in the samples added with antimycin A and D-glucose, the amount of produced lactic acid that is an end product of the glycolytic system actually increased, and occurrence of acceleration of the glycolytic system was verified. Discrimination of Muscle Fiber Type through Autofluorescence Images and Staining of

### Skeletal Muscle Differentiation Induction Samples (FIG. 6)

C2C12 cells were cultured in a 35 mmφ glass bottom dish, and skeletal muscle samples were respectively differentiation induced by using differentiation culture media that are an HS culture medium and an AIM-V culture medium were created (HS differentiation and AIM-V differentiation). For the skeletal muscles of the respective differentiation induction conditions, D-glucose was removed, and then antimycin A+D-glucose were added to create samples. As negative control, for each of the skeletal muscles, only removal of D-glucose was performed, and samples not added with antimycin A+D-glucose were created. For each of the samples, autofluorescence images were captured (B component: the top row in FIG. 6, and G component: the middle row in FIG. 6) as a cell autofluorescence image (B component) of 365 nm excitation/430 nm long pass filter observation and a cell autofluorescence image (G component) of 395 nm excitation/530 nm long pass filter observation. For each of the samples, slow muscle, fast muscle, and nuclei were stained, and the stained images were captured (slow muscle staining: the bottom row in FIG. 6).

### Box-and-Whisker Plot (FIG. 7) of Luminance Values of Autofluorescence at Time of Addition of Reagent to HS Differentiated C2C12 Cells

Autofluorescence luminances of cells in each skeletal muscle region separately by excitation and observation wavelength and RGB, and the luminances in the skeletal muscle region, were set for the luminance values of autofluorescence. For the obtained autofluorescence luminance values of the cells, an autofluorescence change with respect to a skeletal muscle sample differentiation-induced by the HS culture medium that is general differentiation induction was confirmed. A box-and-whisker plot (FIG. 7) was drawn with the ordinate axis representing a B component of an autofluorescence image of 365 nm excitation/430 nm long pass filter observation and a G component of the autofluorescence image of 395 nm excitation/530 nm long pass filter observation. There is a significant difference in G component/B component between the antimycin A+D-glucose added samples and the negative control samples. This demonstrates that an autofluorescence luminance value changes as a result of addition of antimycin A+D-glucose and the change can be quantitatively measured.

### Analysis (FIGS. 8 and 9) of Autofluorescence Information at Time of Addition of Reagent to C2C12 Cells

For the obtained autofluorescence luminance values of the cells, a box-and-whisker plot (FIG. 8) was drawn with the ordinate axis representing a B component of an autofluorescence image of 365 nm excitation/430 nm long pass filter observation and a G component of the autofluorescence image of 395 nm excitation/530 nm long pass filter observation.

As described above, in the HS differentiated samples, there is a significant difference in G component/B component between the antimycin A+D-glucose added samples and the negative control samples, and a similar tendency is also viewed in an AIM-V differentiated samples.

In the negative control samples not added with a reagent, since there is a difference in G component/B component, it is found that cells differentiated to respective muscle fiber types (slow muscle, fast muscle) through differentiation induction by an HS culture medium and an AIM-V culture medium have different autofluorescence spectra and luminances. On the other hand, since the difference is not large, it is difficult to separate muscle fiber types of the cells with only the value of G component/B component. However, when antimycin A+D-glucose are added to each of the HS differentiated sample and the AIM-V differentiated sample, it is found that a difference in G component/B component of autofluorescence is larger than that of the negative control sample.

For the obtained autofluorescence luminance values of the cells, a box-and-whisker plot (FIG. 9) was drawn with the abscissa axis representing a B component of an autofluorescence image of 365 nm excitation/430 nm long pass filter observation and the ordinate axis representing a G component of an autofluorescence image of 395 nm excitation/530 nm long pass filter observation. As in the case of a box-and-whisker plot, in the negative control sample not added with a reagent, a distribution of HS differentiation and a distribution of AIM-V differentiation have about the same inclination, and it is not easy to separate those distributions. On the other hand, in the antimycin A+D-glucose added sample, a distribution of HS differentiation and a distribution of AIM-V differentiation have significantly different inclinations, so cluster separation is easy. From these situations, it becomes easy to discriminate a difference in muscle fiber type by adding a metabolism changing reagent.

With the cell information acquisition methods according to the present disclosure, it is possible to provide the cell information acquisition method that acquires information on a cell by using autofluorescence emitted from the cell regardless of an activation state of the cell.

### Other Embodiments

Embodiment(s) of the present disclosure can also be realized by a computer of a system or apparatus that reads out and executes computer-executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer-executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer-executable instructions. The computer-executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has described exemplary embodiments, it is to be understood that some embodiments of the disclosure are not limited to the disclosed exemplary embodiments. The scope of the following claims is to be accorded the broadest interpretation so as to encompass all such modifications and equivalent structures and functions.

This application claims priority to Japanese Patent Application No. 2023-045118, which was filed on Mar. 22, 2023 and which is hereby incorporated by reference herein in its entirety.

What is claimed is:

1. A cell information acquisition method of acquiring information on a cell by using autofluorescence emitted from the cell, the cell information acquisition method comprising:
a metabolic status changing step of, by changing a metabolic status in the cell, changing at least one of a first autofluorescence luminance indicating a metabolic status of a first metabolic pathway and a second autofluorescence luminance indicating a metabolic status of a second metabolic pathway different from the first metabolic pathway; and
an information acquisition step of, after the metabolic status changing step, acquiring information on the cell by using information on the first autofluorescence luminance and information on the second autofluorescence luminance.

2. The cell information acquisition method according to claim 1, wherein the information on the cell is at least any one of information on a metabolic status of the cell, information on a type of the cell, and information on a phenotype of the cell.

3. The cell information acquisition method according to claim 2, wherein the information on a phenotype of the cell includes information in which a group of the cells is classified into two or more types.

4. The cell information acquisition method according to claim 3, wherein the information in which a group of the cells is classified into two or more types is at least one selected from the group consisting of classification of live cell and dead cell, classification of cancer cell and non-cancer cell, classification of undifferentiated cell and differentiated cell, classification of differentiated cell and dedifferentiated cell, classification of cells in different differentiated states of the same-type differentiated cell type, and classification of epithelial cell and mesenchymal cell of the same-type cancer cells.

5. The cell information acquisition method according to claim 1, wherein the first metabolic pathway and the second metabolic pathway each are any one of a glycolytic system, a TCA cycle, and an electron transfer system.

6. The cell information acquisition method according to claim 1, wherein the metabolic status changing step includes a step of changing at least any one of the metabolic status of the first metabolic pathway and the metabolic status of the second metabolic pathway.

7. The cell information acquisition method according to claim 6, wherein the metabolic status changing step includes a step of accelerating metabolism of the first metabolic pathway and inhibiting metabolism of the second metabolic pathway.

8. The cell information acquisition method according to claim 6, wherein the metabolic status changing step includes an addition step of adding a reagent having a component that changes at least any one of the metabolic status of the first metabolic pathway and the metabolic status of the second metabolic pathway.

9. The cell information acquisition method according to claim 8, wherein the addition step includes a step of adding a reagent having a component that accelerates metabolism of the first metabolic pathway and that inhibits metabolism of the second metabolic pathway.

10. The cell information acquisition method according to claim 8, wherein the reagent includes at least one selected from the group consisting of a glycolytic system accelerator, a glycolytic system inhibitor, a TCA cycle accelerator, a TCA cycle inhibitor, an electron transfer system accelerator, and an electron transfer system inhibitor.

11. The cell information acquisition method according to claim 8, wherein the reagent contains D-glucose and antimycin A.

12. The cell information acquisition method according to claim 8, wherein the reagent is noninvasive for the cell.

13. The cell information acquisition method according to claim 8, wherein, when the reagent is removed, the reagent allows a metabolic status to reversibly change to a metabolic status before the reagent is added to the cell.

14. The cell information acquisition method according to claim 8, further comprising a step of adding the reagents different from each other to respectively a plurality of samples each including the cell and acquiring an autofluorescence luminance after the addition.

15. The cell information acquisition method according to claim 1, further comprising a step of, before the metabolic status changing step, acquiring information on the cell in a state where metabolism of the cell remains unchanged.

16. The cell information acquisition method according to claim 1, wherein the metabolic status changing step is a step capable of reversibly changing a metabolic status to a metabolic status before the metabolic status changing step.

17. The cell information acquisition method according to claim 1, further comprising a step of culturing the cell after the information acquisition step.

18. A non-transitory computer-readable medium storing computer-executable instructions that, when executed by one or more computers, cause the one or more computers to execute a cell information acquisition method, the cell information acquisition method comprising:
a metabolic status changing step of, by changing a metabolic status in the cell, changing at least one of a first autofluorescence luminance indicating a metabolic status of a first metabolic pathway and a second autofluorescence luminance indicating a metabolic status of a second metabolic pathway different from the first metabolic pathway; and
an information acquisition step of, after the metabolic status changing step, acquiring information on the cell by using information on the first autofluorescence luminance and information on the second autofluorescence luminance.
